(19) United States
(12) Patent Application Publication
(10) Pub. No.: US 2016/0200640 A1
(43) Pub. Date: Jul. 14, 2016

(54) RADIOACTIVE AGENT SYNTHESIS DEVICE AND METHOD

(71) Applicant: HITACHI, LTD., Tokyo (JP)

(72) Inventors: Naomi MANRI, Tokyo (JP); Norihito KUNO, Tokyo (JP)

(21) Appl. No.: 14/912,423

(22) PCT Filed: Aug. 28, 2013
(86) PCT No.: PCT/JP2013/072948

§ 371 Date: Feb. 17, 2016

(51) Int. Cl. C07B 59/00; B01J 19/24; A61K 51/08

(52) U.S. Cl. CPC C07B 59/008; A61K 51/081; B01J 19/24; B01J 2219/24; B01J 2219/00049

(57) ABSTRACT

In a reaction between a biomolecule and a radioactive agent precursor, the amount of a component of a radioactive agent precursor before the reaction and the amount of a component contained in a reaction mixture in a reaction process in which the reaction is performed at a predetermined biomolecule concentration are measured, a reaction rate constant is calculated from the information of the obtained amounts of the components, a reaction rate constant when the biomolecule concentration is changed is calculated, a reaction time, a specific radioactivity, and a radioactivity level of an objective substance at each biomolecule concentration are predicted, and synthesis is performed under a reaction condition under which the highest specific radioactivity is obtained among the reaction conditions satisfying the synthesis requirements.

### TECHNICAL FIELD

The present invention relates to radioactive agent synthesis device and method, and particularly relates to efficient synthesis in a radiolabeling reaction of a biomolecule.

### BACKGROUND ART

Recently, image diagnosis and evaluation of pharmacokinetics of drugs by positron emission tomography (PET) have been actively performed. PET is a technique for performing diagnosis by administering an agent (radioactive agent) containing a radioisotope which emits positron by intravenous injection or inhalation and imaging the biodistribution thereof, and has a characteristic that the information of the physiological and biochemical functions of organs is obtained. PET enables the state of cell activity to be observed by an image unlike the conventional tests for observing shape such as CT or MRI, and therefore has been utilized for diagnosis of the causes or symptoms of cancer, heart diseases and brain diseases. Further, PET also has an advantage that systemic lesions can be examined by a single administration. For this reason, PET has been used not only in diagnosis, but also in the field of drug discovery research in which pharmacokinetics of new drugs is evaluated.

Examples of a positron nuclide to be used in PET diagnosis include ¹¹C, ¹³N, ¹⁵O, and ¹⁸F. The half-lives of these nuclides are 20.4 min, 9.97 min, 2.04 min, and 109.8 min, respectively, and very short. Therefore, these nuclides cannot be stored, and it is necessary to synthesize at the site, and therefore, it has been required that the nuclide be synthesized in a short time with high efficiency.

As radioactive agents specifically accumulated at a disease site, many peptides and proteins have been studied. In particular, along with the popularization of antibody preparations and biological drugs, the usefulness of a protein can be determined by radiolabeling the protein and evaluating the pharmacokinetics thereof, and therefore, protein labeling has been attracting attention.

Protein radiolabeling is performed by reacting a protein with a radioactive agent precursor. Many radioactive agent precursors have been developed, and N-succinimidyl-4-[¹⁸F]fluorobenzoate ([¹⁸F]SFB) which radiolabels a lysine residue or an N-terminal amine or the like is well known (see NPL: 1).

In a labeling reaction using [¹⁸F]SFB, as a protein concentration increases, the ratio (labeling ratio) of [¹⁸F]SFB to be used for protein labeling increases and the synthesis time decreases (see NPL: 2). However, there is a limit to the production amount, and therefore, fora radioactive agent precursor, only a small amount of which can be used for the reaction, a protein is used in a large amount. As a result, the specific radioactivity (the radioactivity level per unit weight) of the synthesized radioactive agent decreases, and most of the component results in an unlabeled protein. When the synthesized radioactive agent is administered to a living body, most of the protein accumulated in a target region is the unlabeled protein, and thus, a problem arises that the detection sensitivity decreases. On the other hand, when the reaction is performed under a low protein concentration condition, the labeling ratio of the protein is low, and it takes time for synthesis, and therefore, due to radioactive decay, the specific radioactivity decreases. Liu et al. performed an experiment actually under several protein concentration conditions, and among the conditions, a protein concentration condition at which the highest specific radioactivity is obtained has been determined (see NPL: 2). It is difficult to separate and purify a labeled protein after a labeling reaction, and therefore, it is necessary to synthesize a radioactive agent with a high specific radioactivity by setting a suitable reaction condition.

It is known that the synthesis time or the labeling ratio having an influence on the specific radioactivity varies depending on the type or purity of a radioactive agent precursor, or the type or concentration of a protein or a peptide (see NPL: 3).

Further, there is also a provision regarding a radioactivity level necessary for PET imaging. For example, in the case of [¹⁸F]FDG, it is provided that the radioactivity level is from 185 to 444 MBq (3 to 7 MBq/kg) for 2D data collection, from 111 to 259 MBq (2 to 5 MBq/kg) for 3D data collection (see NPL: 4). Due to this, in the synthesis of a radioactive agent, not only a high specific radioactivity, but also ensuring of a necessary radioactivity level for PET imaging is an important requirement for the synthesis of a radioactive agent.

### CITATION LIST

### Non Patent Literature

NPL 1: P. W. Miller, N.J. Long, R. Vilar, and A. D. Gee: Synthesis of 11C, 18F, 15O, and 13N Radiolabels for Positron Emission Tomography: Angew. Chem. Int. Ed., 47, 8998-9033 (2008)

NPL 2: K. Liu, E. J. Lepin, M. Wang, F. Guo, W. Lin, Y. Chen, S. J. Sirk, S. Olma, M. E. Phelps, X. Zhao, H. Tseng, R. Michael van Dam, A. M. Wu, and C. Shen: Microfluidic-based 18F-labeling of Biomolecules for ImmunoPET: Mol. Imaging, 10, 168-177 (2011)

NPL 3: P. Johnstrom, J. C. Clark, J. D. Pickard, and A. P. Davenport: Automated synthesis of the generic peptide labelling agent N-succinimidyl 4-[18F]fluorobenzoate and application to 18F-label the vasoactive transmitter urotensin-II as a ligand for positron emission tomography: Nuclear Medicine and Biology., 35, 725-731 (2008)

NPL 4: Guidelines for clinical use of FDG PET, PET/CT 2010 (April, 2010, The Japanese Society of Nuclear Medicine)

### SUMMARY OF INVENTION

### Technical Problem

The synthesis of a radiolabeled protein is performed by reacting a protein with a radioactive agent precursor. For PET imaging with high sensitivity, it is necessary to synthesize a radioactive agent with a high specific radioactivity. It is difficult to separate an unlabeled protein from a labeled protein after synthesis, and therefore, a reaction condition capable of synthesizing a radioactive agent with a high specific radioactivity during synthesis should be set. Further, also a radioactivity level necessary for PET imaging should be ensured.

The specific radioactivity or the radioactivity level of a protein component after synthesis is determined by a synthesis time or a labeling ratio, however, the synthesis time or the labeling ratio varies depending on the type or purity of a radioactive agent precursor, or the type or concentration of a protein. Due to this, it is difficult to predict the specific radioactivity or the radioactivity level of a protein component after synthesis.

It is possible to measure the specific radioactivity or the radioactivity level of a protein component after synthesis by performing a preliminary examination before synthesis using a radioactive agent precursor and a protein to be used in the synthesis at several protein concentration conditions. However, if it takes time for the preliminary examination, due to the effect of radioactive decay, the specific radioactivity or the radioactivity level after synthesis decreases. Further, it is not necessarily the case that a reaction condition under which the highest specific radioactivity is obtained is included in the protein concentration conditions for which the preliminary examination was performed. Further, unless the synthesis is stopped at an appropriate time, the specific radioactivity or the radioactivity level of the protein component after synthesis decreases.

In light of this, an object of the invention is to synthesize a radioactive agent under a protein concentration condition at which the specific radioactivity is high while securing a radioactivity level of an objective substance.

Further, the invention is directed to the synthesis a radioactive agent without decreasing the specific radioactivity.

### Solution to Problem

A radioactive agent synthesis device according to the invention is preferably configured as a radioactive agent synthesis device characterized by including: a reaction vessel in which at least a radioactive agent precursor solution and a protein solution are placed and a synthesis reaction is performed; a measurement section which measures the amount of a component of a radioactive agent precursor before the reaction and the amount of a component of a reaction mixture in the middle of the reaction; a processing section which performs synthesis processing including processing in which a reaction rate constant is calculated from the measurement information of the amounts of the components, a reaction rate constant when a biomolecule concentration is changed is calculated, and a reaction time, a specific radioactivity, and a radioactivity level of an objective substance at each biomolecule concentration are calculated; a display section which displays the result of the synthesis processing by the processing section; and an input section from which a condition for the synthesis processing is input, wherein the processing section uses the result of the first synthesis processing obtained by the processing section as a synthesis condition for second synthesis processing to be performed thereafter along with the condition to be input from the input section.

A radioactive agent synthesis method according to the invention is preferably a radioactive agent synthesis method for synthesizing a radioactive agent using a radioactive agent precursor, and is configured as a radioactive agent synthesis method characterized in that first synthesis is performed according to preset first synthesis conditions including a radioactivity level, a synthesis volume, and a protein concentration, and the data of a specific radioactivity and a reaction completion time determined by the first synthesis are used for setting the conditions for second synthesis to be performed thereafter.

In a preferred embodiment, the first synthesis includes a step of measuring the amount of a component of a radioactive agent precursor before the reaction, a step of measuring the amount of a component of a reaction mixture in the middle of the reaction, a step of calculating a reaction rate constant from the measurement information of the amounts of the components, a step of calculating a reaction rate constant when a biomolecule concentration is changed, a step of calculating a reaction time, a specific radioactivity, and a radioactivity level of an objective substance at each biomolecule concentration, and a step of selecting a reaction condition satisfying the synthesis condition, and the second synthesis is performed under the selected reaction condition.

### Advantageous Effects of Invention

According to the invention, it becomes possible to synthesize a radioactive agent under a protein concentration condition under which the specific radioactivity is high while securing a radioactivity level of an objective substance. Further, the reaction can be completed at an optimal time, and therefore, it becomes possible to synthesize a radioactive agent without decreasing the specific radioactivity. According to this, in the synthesis of a radioactive agent, the time is reduced and the efficiency of the synthesis is improved.

### BRIEF DESCRIPTION OF DRAWINGS

FIG. 1 is a view showing a configuration example of a radioactive agent synthesis device in one embodiment.

FIG. 2 is a view showing the operation of a control flow for a radioactive agent in one embodiment.

FIG. 3 is a view showing one example of LC analysis conditions.

FIG. 4 is a view showing an experimental diagram of a liquid chromatogram.

FIG. 5 is a view illustrating the synthesis pathway using [¹⁸F]SFB and a protein.

FIG. 6 shows experimental diagrams of liquid chromatograms.

FIG. 7 is a view showing that the predicted curves (broken lines) and the experimental values of [¹⁸F]SFB, [¹⁸F]FBzA, and [¹⁸F]BSA in a reaction between [¹⁸F]SFB and BSA (0.25 mg/mL) coincide with each other.

FIG. 8 is a view showing that the predicted curves (broken lines) and the experimental values of [¹⁸F]SFB, [¹⁸F]FBzA, and [¹⁸F]BSA in a reaction between [¹⁸F]SFB and BSA (2.5 mg/mL) coincide with each other.

FIG. 9 is a view showing a relationship between a radioactivity level of a protein component at a half -life of 110 minutes and a reaction time (A), a relationship between a protein concentration and a reaction completion time (B), a relationship between a protein concentration and a radioactivity level of a protein component at completion of the reaction (C) and a relationship between a protein concentration and a specific radioactivity of a protein component at completion of the reaction (D).

FIG. 10 is a view showing a relationship between a radioactivity level of a protein component at a half-life of 5 minutes and a reaction time (A), a relationship between a protein concentration and a reaction completion time (B), a relationship between a protein concentration and a radioactivity level of a protein component at completion of the reaction (C) and a relationship between a protein concentration and a specific radioactivity of a protein component at completion of the reaction (D).

FIG. 11 is a view showing the operation of a processing flow of a processing section 170 in one embodiment.

FIG. 12 is a view showing one example of a setting screen for LC analysis.

FIG. 13 is a view showing one example of a setting screen for test synthesis.

FIG. 14 is a view showing one example of a setting screen for actual synthesis.

### DESCRIPTION OF EMBODIMENTS

Hereinafter, preferred embodiments of the invention will be described with reference to the drawings.

### Configuration of Device

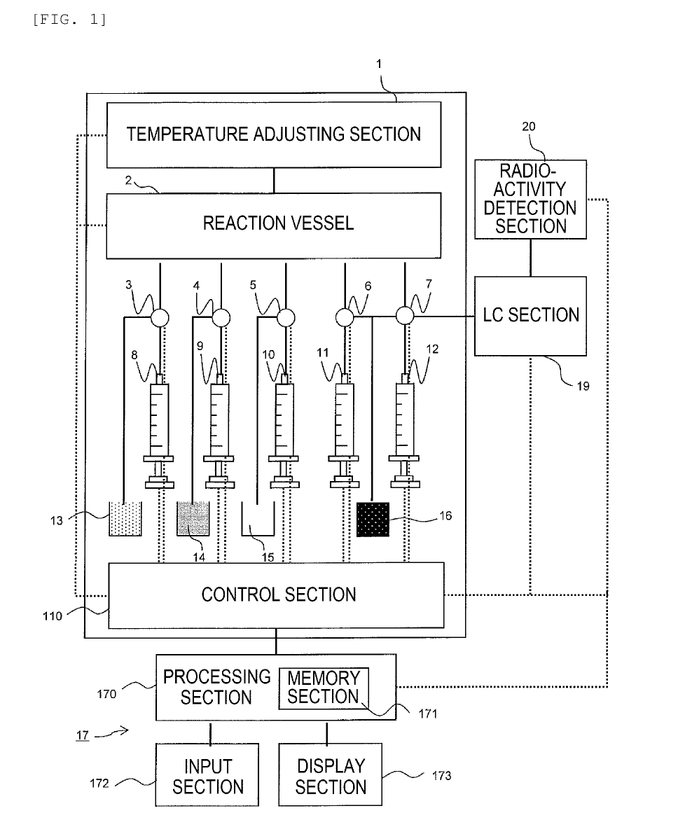

FIG. 1 shows a configuration example of a radioactive agent synthesis device.

This synthesis device includes storage sections storing a plurality of solutions, and a protein solution storage section 13 stores a protein solution, a buffer solution storage section 14 stores a buffer solution, a water storage section 15 stores water, and a radioactive agent precursor solution storage section 16 stores a radioactive agent precursor solution.

A syringe 8 sucks the protein solution from the protein solution storage section 13 through a valve 3 and introduces the sucked protein solution into a reaction vessel 2 through the valve 3. A syringe 9 sucks the buffer solution from the buffer solution storage section 14 through a valve 4 and introduces the sucked buffer solution into the reaction vessel 2 through the valve 4. A syringe 10 sucks water from the water storage section 15 through a valve 5 and introduces the sucked water into the reaction vessel 2 through the valve 5. A syringe 11 sucks the radioactive agent precursor solution from the radioactive agent precursor solution storage section 16 through a valve 6 and introduces the sucked radioactive agent precursor solution into the reaction vessel 2 through the valve 6. A syringe 12 sucks the radioactive agent precursor solution from the radioactive agent precursor solution storage section 16 through a valve 7 and transfers the sucked radioactive agent precursor solution to an LC section 19 through the valve 7. Further, the syringe 12 sucks a reaction mixture from the reaction vessel 2 through the valve 7 and transfers the sucked reaction mixture to the LC section 19 through the valve 7. By the operation of the syringes 8 to 11, the protein, the radioactive agent precursor, and the buffer solution can be adjusted to a desired concentration, and are reacted to each other in the reaction vessel 2. The reaction vessel 2 includes a stirrer for mixing the solution and an agitator for performing bubbling or the like with nitrogen gas or the like. The temperature of the reaction vessel 2 is controlled by a temperature adjusting section 1.

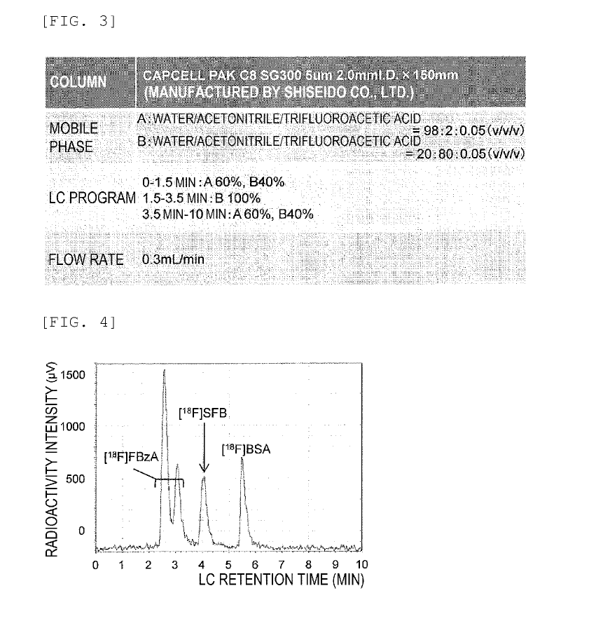

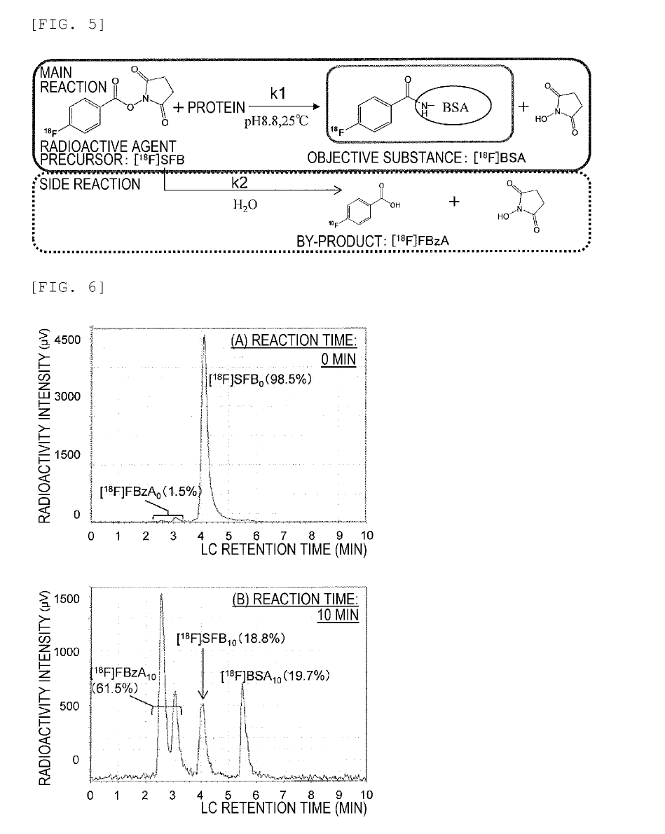

The LC section 19 performs LC analysis of a product obtained by the reaction in the reaction vessel 2. A radioactivity detection section 20 detects the radioactivity of the product analyzed by the LC section 19. The LC section 19 and the radioactivity detection section 20 may be collectively called “measurement section”. Here, in the LC analysis by the LC section 19, an elution condition for separating the radioactive agent precursor, the radiolabeled protein, and if present, a by-product is determined in advance. For example, when LC analysis was performed under the elution conditions shown in FIG. 3 for a reaction mixture at a reaction time of 10 min in a labeling reaction of a protein with [¹⁸F]SFB using bovine serum albumin (BSA) (a reaction between [¹⁸F]SFB and BSA (0.25 mg/mL) at pH 8.8 and 25° C.), a liquid chromatogram shown in FIG. 4 was obtained. The following three components: [¹⁸F]SFB, BSA radiolabeled with [¹⁸F]SFB (hereinafter abbreviated as “[¹⁸F]BSA”), and [¹⁸F]fluorobenzoic acid (hereinafter abbreviated as “[¹⁸F]FBzA”) which is a by-product produced by hydrolysis of [¹⁸F]SFB could be separated.

In one example, in the liquid chromatogram shown in FIG. 4, the data were obtained using a high-performance liquid chromatograph. Recently, a high- throughput ultra-high-performance liquid chromatograph is available, and by using this device, a data acquisition time can be reduced. Alternatively, another analysis method may be used as long as the amount of a component can be measured.

A control section 110 controls the respective sections such as the temperature adjusting section 1, the agitator of the reaction vessel 2, the valves 3 to 7, the syringes 8 to 12, the LC section 19, and the radioactivity detection section 20. The information of the liquid chromatogram obtained from the radioactivity detection section 20 is provided to an information processing device 17.

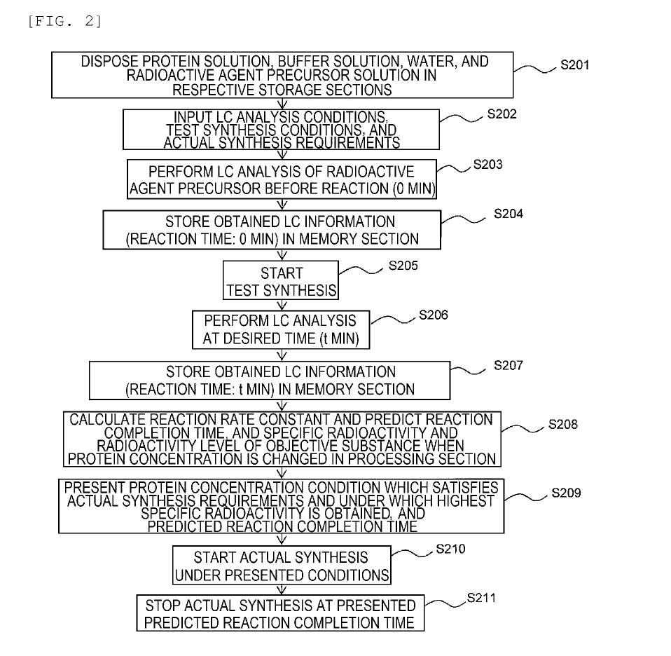

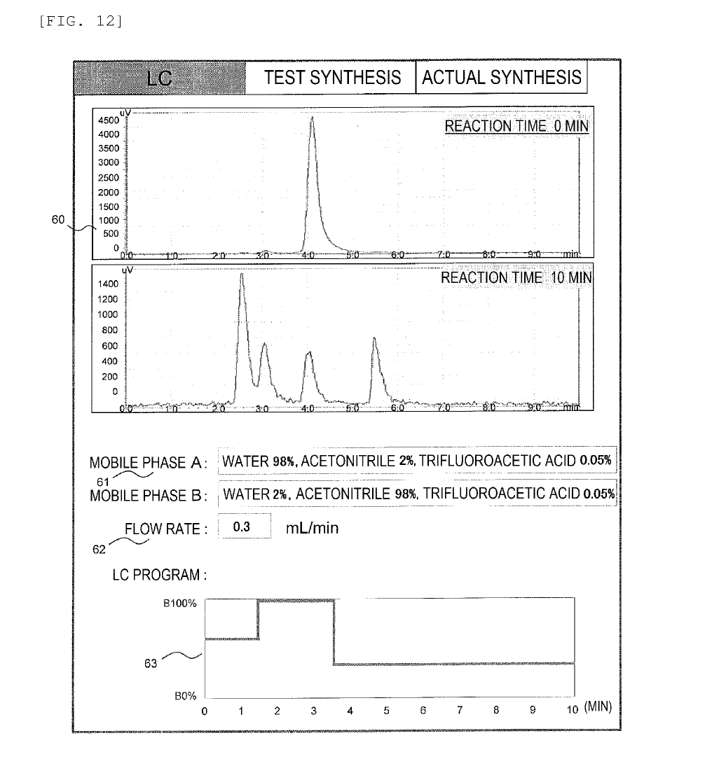

The information processing device 17 is, for example, a personal computer (hereinafter simply referred to as “PC”), and includes a processing section 170 which performs data processing for executing a program by a processor, a memory section 171 such as a hard disk or a memory for storing data or a program, an input section 172 such as a keyboard or a mouse from which data can be input, and a display section 173 such as a liquid crystal display for displaying the information on a screen. The processing section 170 performs data processing and obtains data, for example, a reaction rate constant, a protein concentration condition, and the like by executing a program. Further, the processing section 170 creates a graph showing a relationship between a protein concentration and a predicted reaction completion time, a graph showing a relationship between a protein concentration and a radioactivity level obtained at completion of the reaction, a graph showing a relationship between a protein concentration and a specific radioactivity, and the like. The processing by the processing section 170 will be described later with reference to FIGS. 2 and 11.

The display section 173 displays the following data on the screen: an elution condition, a flow rate, and the like for LC the concentration and volume of a protein placed in the protein solution storage section 13, the type of a radioactive agent precursor, the radioactivity level and volume of a radioactive agent precursor placed in the radioactive agent precursor solution storage section 16, the type and concentration of a buffer solution, a synthesis volume, a radioactivity level, a protein concentration, a reaction temperature, timing for performing LC analysis (a reaction time at which LC analysis is performed), and an LC analysis amount for the below-mentioned test synthesis, a radioactivity level of an objective substance, a radioactivity level to be used, a synthesis volume, and a buffer solution concentration, which are the below-mentioned actual synthesis requirements, a reaction rate constant, a graph showing a relationship between a protein concentration and a predicted reaction completion time, a graph showing a relationship between a protein concentration and a radioactivity level obtained at completion of the reaction, and a graph showing a relationship between a protein concentration and a specific radioactivity obtained by the below-mentioned calculation processing, a protein concentration condition, a reaction time, a predicted radioactivity level of an objective substance, a predicted specific radioactivity, and the like deduced from the calculation results. The input section 172 is used for performing operation input for inputting or displaying data of various conditions for the above-mentioned data processing by the operation of a user.

Incidentally, in the example shown in FIG. 1, the operations of sucking and discharging a solution are performed using a syringe, but may be performed by another means as long as the solution can be transferred. In the case where washing of the syringe is needed, the washing may be performed by providing a washing solution storage section. Further, a disposable syringe may be used.

In this device, the reaction is performed by mixing solutions, however, the reaction may be performed by adding a liquid to the reaction vessel containing a protein and a radioactive agent precursor, both of which are dried to a solid.

In this device, the protein solution storage section 13 and the radioactive agent precursor solution storage section 16 do not have a temperature adjusting function, but may have a temperature adjusting function.

### Synthesis Control Operation

FIG. 2 is a flow showing the operation of control of synthesis of a radioactive agent.

First, a user disposes a protein solution, a buffer solution, water, and a radioactive agent precursor solution in the following respective storage sections: the protein solution storage section 13, the buffer solution storage section 14, the water storage section 15, and the radioactive agent precursor solution storage section 16 (S201), respectively. The buffer solution is adjusted to a desired pH in advance.

Subsequently, the user inputs data of LC analysis conditions (a flow rate, an LC elution condition, and the like, see FIG. 3), test synthesis conditions (a synthesis volume, a radioactivity level, a protein concentration, a reaction temperature, a timing and a volume for LC analysis, and the like), actual synthesis requirements (a synthesis volume, a buffer solution concentration, a radioactivity level to be used, a radioactivity level of an objective substance, a reaction temperature, and the like) from the input section 172 (S202). The actual synthesis requirements may be input after the test synthesis.

Subsequently, by driving the syringe 12, the radioactive agent precursor solution of a preset volume for LC analysis is sucked from the radioactive agent precursor solution storage section 16, and LC analysis at a reaction time of 0 min is performed (S203). The obtained information of the liquid chromatogram is stored in the memory section 171 (S204).

Subsequently, the user starts test synthesis by driving the syringes 8 to 11 to mix the protein solution, the buffer solution, water, and the radioactive agent precursor solution so that a reaction is performed under preset test synthesis conditions (S205). By driving the syringe 12 at a time (t min) set in the test synthesis conditions, the reaction mixture of a preset volume for LC analysis is sucked from the reaction vessel 2, and LC analysis is performed (S206). Incidentally, the LC analysis may be performed at a desired time, however, it is necessary to obtain data in the middle of the reaction. Further, it is desirable to perform the LC analysis a plurality of times for reducing the error of the below-mentioned reaction rate constant. The obtained information of the liquid chromatogram is stored in the memory section 171 (S207).

Subsequently, a reaction rate constant is calculated by the processing of the processing section 170, and a reaction completion time, and a specific radioactivity and a radioactivity level of an objective substance when a protein concentration is changed are predicted (S208). Incidentally, a detailed processing operation of the processing section 170 will be described later with reference to FIG. 11. As a result of this prediction processing, a protein concentration condition which satisfies the actual synthesis requirements and under which the highest specific radioactivity is obtained, a predicted reaction completion time, a predicted radioactivity and a predicted specific radioactivity of the objective substance are presented, that is, displayed on the screen of the display section 173 (S209).

Thereafter, the actual synthesis is started by driving the syringes 8 to 11 and mixing the protein solution, the buffer solution, the radioactive agent precursor solution, and water such that the presented reaction conditions are satisfied (S210). Then, the actual synthesis is stopped at the presented predicted reaction completion time (S211).

By utilizing this control flow, it is possible to predict a reaction when the protein concentration is changed from the result of the test synthesis, and to perform the actual synthesis under a condition under which the highest specific radioactivity is obtained among the reaction conditions satisfying the requirement condition for the objective substance.

### Calculation Method using Mathematical Formula

Here, it is explained that it is possible to predict a reaction when a protein concentration is changed by using a protein labeling reaction with a radioactive agent precursor [¹⁸F]SFB as an example and calculating a reaction rate constant from the LC experimental data by the processing section 170. Incidentally, the radioactive agent precursor is not limited to [¹⁸F]SFB, and another radioactive agent precursor may be used. Further, the mathematical formula is sometimes different depending on the reaction, however, also in such a case, calculation processing suitable for the reaction may be performed.

FIG. 5 shows the synthesis pathway of a protein labeling reaction using [¹⁸F]SFB. There are two reaction pathways as follows:

a main reaction pathway in which [¹⁸F]SFB reacts with a protein to produce a [¹⁸F] protein; and a side reaction in which [¹⁸F]SFB reacts with water to produce [¹⁸F]FBzA. When the reaction rate constants are represented by k1 and k2, respectively, in a primary reaction A→C (reaction rate constant: k′), the reaction rate v is represented by −d [A]/dt=k′ [A], and in a secondary reaction A+B→C (reaction rate constant: k′), the reaction rate v is represented by −d[A]/dt=k′[A] [B].

The side reaction in which [¹⁸F]SFB reacts with a water molecule which is present in a large excess amount with respect to [¹⁸F]SFB can be regarded as a primary reaction, and therefore, the degradation rate of [¹⁸F]SFB is represented as follows: −d [[¹⁸F]SFB]/dt=k1[[¹⁸F]SFB] [protein]+k2[[¹⁸F]SFB]=(k2+k1[protein])[[¹⁸F]SFB] in consideration of the main reaction and the side reaction. A protein generally has a plurality of lysine residues, and even if the protein is labeled with [¹⁸F]SFB at one site, the protein can be further labeled with [¹⁸F]SFB. For example, bovine serum albumin (BSA) has 60 lysine residues. Therefore, since it can be considered that the protein concentration does not change during the reaction, k2+k1[protein] becomes a constant, and the reaction is regarded as a primary reaction represented by −d[[¹⁸F]SFB]/dt=k[[¹⁸F]SFB](k=k1[protein]+k2). It is found that when the protein concentration is changed, also k is changed. The concentration of [¹⁸F]SFB at a reaction time of t [[¹⁸F]SFBₜ] can be represented by the formula (1) when the initial concentration is represented by [[F]SFB₀].

[Math. 1]

[[¹⁸F]SFBₜ]=[[¹⁸F]SFB₀]·e^{-kt} (1)

That is, k is represented by the following formula, and it is possible to calculate k using the initial concentration [[¹⁸F]SFB₀] and the concentration at a reaction time of t [[¹⁸F]SFBₜ] obtained by the LC analysis.

[Math. 2]

k=−In[[[¹⁸F]SFBₜ]/[[¹⁸F]SFB₀]]/t (2)

Further, the change in concentration of [¹⁸F]protein and [¹⁸F]FBzA can be represented by d[[¹⁸F]protein]/dt=k1[protein] [[¹⁸F]SFB] and d[[¹⁸F]FBzA]/dt=k2[[¹⁸F]SFB], respectively, and therefore, the following relationship is established, and the ratio of the concentration of [¹⁸F]FBzA to [¹⁸F ]protein is always constant during the reaction.

[Math. 3]

d[[¹⁸F]protein]/d[[¹⁸F]FBzA]=k₁[protein]/k₂ (3)

From the formula (3) and the formula: k=k1[protein]+k2, the following formulae can be deduced.

[Math. 4]

[[¹⁸F]proteinₜ]/[[¹⁸F]proteinₜ]+[[¹⁸F]FBzAₜ]]=k₁[protein]/k (4)

[Math. 5]

[[¹⁸F]FBzAₜ]/[[¹⁸F]proteinₜ]+[[¹⁸F]FBzAₜ]=k₂/k (5)

The concentration is estimated from k calculated from the formula (2), and the peak areas of [[¹⁸F]proteinₜ] and [[¹⁸F]FBzAₜ] obtained by the LC analysis, and by using the formulae (4) and (5), k1[protein] and k2 can be calculated. Incidentally, the same result is obtained even when the abundance (%) of each peak is used in place of the concentration.

Further, [[¹⁸F]proteinₜ]and [[¹⁸F]FBzAₜ] at a reaction time of t can be represented by the following formulae, respectively, and therefore, if k1[protein] and k2 can be calculated, the progress of the reaction can be predicted.

[Math. 6]

[[¹⁸F]proteinₜ]=k₁[protein]/k(1−e^{-kt}) (6)

[Math. 7]

[[¹⁸F]FBzAₜ]=k₂/k(1−e^{-kt}) (7)

Accordingly, by measuring the amount of a component before the reaction and in the reaction process, k1[protein] and k2 can be calculated, and the progress of the reaction can be predicted. Further, k1[protein] is proportional to the protein concentration, and k2 does not change, and therefore, it is also possible to predict the progress of the reaction when the protein concentration is changed.

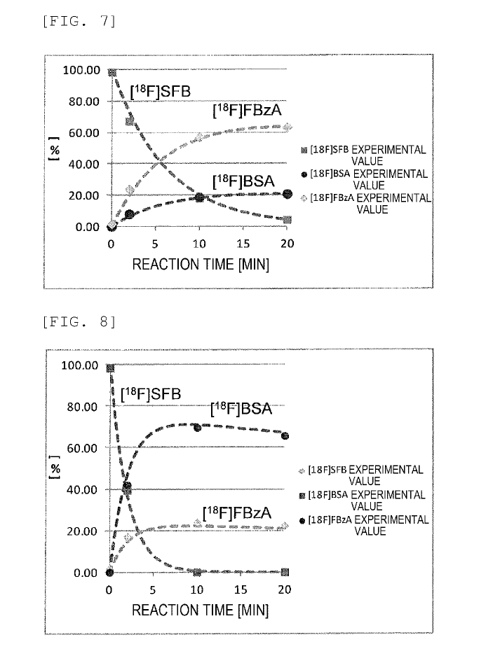

Here, an explanation is made using experimental data. The explanation is made using a reaction in which a protein is labeled with ¹⁸F by mixing BSA (0.5 mg/mL) dissolved in a 125 mM borate buffer solution (pH 8.8) and [¹⁸F]SFB dissolved in 20% acetonitrile in equal amounts as an example. FIG. 6(A) is a liquid chromatogram obtained by LC analysis (reaction time: 0 min) of [¹⁸F]SFB dissolved in 20% acetonitrile. FIG. 6 (B) is a liquid chromatogram obtained by performing LC analysis of part of the reaction mixture after 10 minutes from the start of the reaction of [¹⁸F]SFB and BSA. The LC analysis was performed under the conditions shown in FIG. 3.

When the reaction time was 0 minutes, [¹⁸F]FBzA having an LC retention time ranging from 2.5 minutes to 3.5 minutes and [¹⁸F]SFB having an LC retention time of 4.5 minutes were detected, and when expressed in terms of percentage, [[¹⁸F]SFB₀] was 98.5%, and [[¹⁸F]FBzA₀] was 1.5%. It is found that 1.5% of [¹⁸F]SFB has been hydrolyzed before it is reacted with a protein (FIG. 6(A)). When the reaction time was 10 minutes, [¹⁸F]FBzA having an LC retention time ranging from 2.5 minutes to 3.5 minutes, [¹⁸F]SFB having an LC retention time of 4.5 minutes, and [¹⁸F]BSA having an LC retention time of 5.6 minutes were detected, and when expressed in terms of percentage, [¹⁸F]FBzA₁₀ was 61.5% [¹⁸F]SFB₁₀ was 18.8%, and [¹⁸F]BSA₁₀ was 19.7%. Since [¹⁸F]FBzA was present in an amount of 1.5% of the total peak area before the reaction from the data at a reaction time of 0 min, 60.0% obtained by subtracting 1.5% from 61.5% [[¹⁸F]FBzA₁₀] at a reaction time of 10 min is the percentage of the [¹⁸F]FBzA produced by the reaction. That is, at a reaction time of 10 min, [¹⁸F]SFB, which was present at 98.5% before the reaction decreased to 18.8%, and [¹⁸F]FBzA was produced at 60.0%, and BSA labeled with ¹⁸F ([¹⁸F]BSA) was produced at 19.7%. By using these values, k1[BSA] and k2 are calculated.

First, k is calculated using the formula (2) as follows: k=−ln(18.8/98.5)/10=0.166. Subsequently, the calculated k, [[¹⁸F]FBzA₁₀]:60.0, and [[¹⁸F]BSA₁₀]: 19.7 are substituted in the formula (4) as follows: 19.7/(60.0+19.7)=k1[BSA]/0.166, so that k1[BSA] is calculated as follows: k1[BSA]=0.041. In the same manner, from the formula (5), k2 is calculated as follows: k2=0.125. Alternatively, from the following formula: k=k1[protein]+k2, by substituting the calculated k and k1[BSA], k2=0.125 may be calculated.

By substituting the calculated k1[BSA] and k2 in the formulae (1), (6), and (7), it is possible to represent a relationship between a reaction time t and the ratio of each of [¹⁸F]SFB, [¹⁸F]BSA, and [¹⁸F]FBzA. However, these formulae are relational formulae in consideration of only the reaction itself, and in fact, radioactive decay occurs. When considering radioactive decay, the formulae (1), (6), and (7) become the formulae (8), (9), and (10), respectively.

The formulae are expressed in percentage.

[Math. 8]

[[¹⁸F]SFBₜ]=100×[[¹⁸F]SFB₀]·e^{-kt}×0.5^{t/half-life} (8)

[Math. 9]

[[¹⁸F]BSAₜ]=100×k₁[BSA]/k(1−e^{-kt})×0.5^{t/half-life} (9)

[Math. 10]

[[¹⁸F]FBzAₜ]=100×k₂/k(1−e^{-kt})×0.5^{t/half-life} (10)

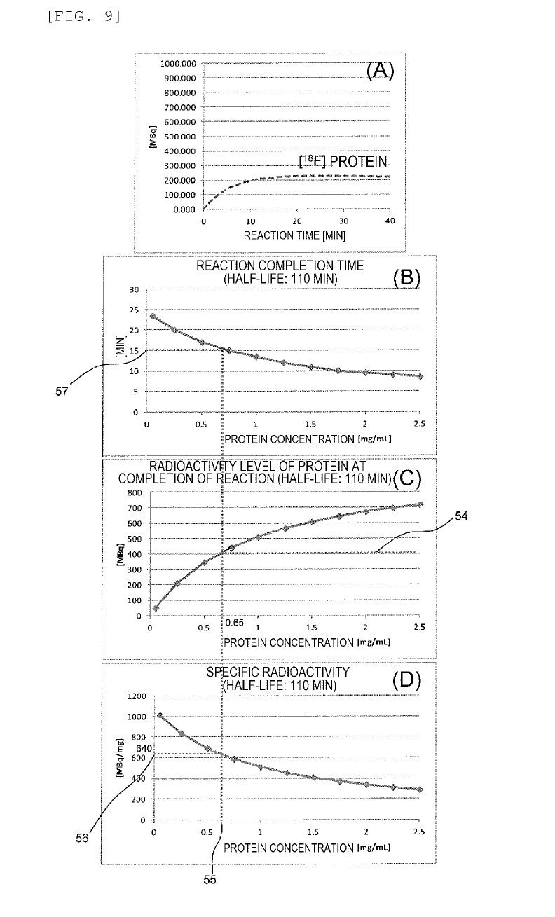

The calculated k1[BSA] and k2 are substituted in the formulae (8), (9), and (10), and graphs are created as in FIG. 7. The half-life was set to 110. However, in the LC analysis before the reaction (reaction time: 0 min), [[¹⁸F]SFB₀] was 98.5%, and therefore, the graphs were created by changing 100 in the right side of each of the formulae (8), (9), and (10) to 98.5. Further, [[¹⁸F]FBzA₀] was 1.5% at a reaction time of 0 min, and therefore, the graphs were created by adding 1.5 to the right side of the formula (10). When the experimental data obtained at reaction times of 2, 10, and 20 min were plotted on the prediction curves, it could be confirmed that the data coincide with the curves of the formulae (8), (9), and (10). The experimental data were obtained as follows: each of the peak areas of [¹⁸F]SFB, [¹⁸F]BSA, and [¹⁸F]FBzA from the LC data was converted to a percentage, and multiplied by 0.5^{t/half-life} in consideration of radioactive decay. For t, the reaction time was substituted. Accordingly, in this graph, the amount of ¹⁸F at a reaction time of 0 min is taken as 100%, and therefore, as the reaction time is increased, the sum of the amounts of the three components is decreased to a value smaller than 100% due to radioactive decay.

Subsequently, by using k1[BSA]=0.041 and k2=0.125 obtained by the reaction of BSA (0.25 mg/mL), a reaction of BSA (2.5 mg/mL) in which the BSA concentration was increased to 10 times is predicted, and compared with the actual experimental data.

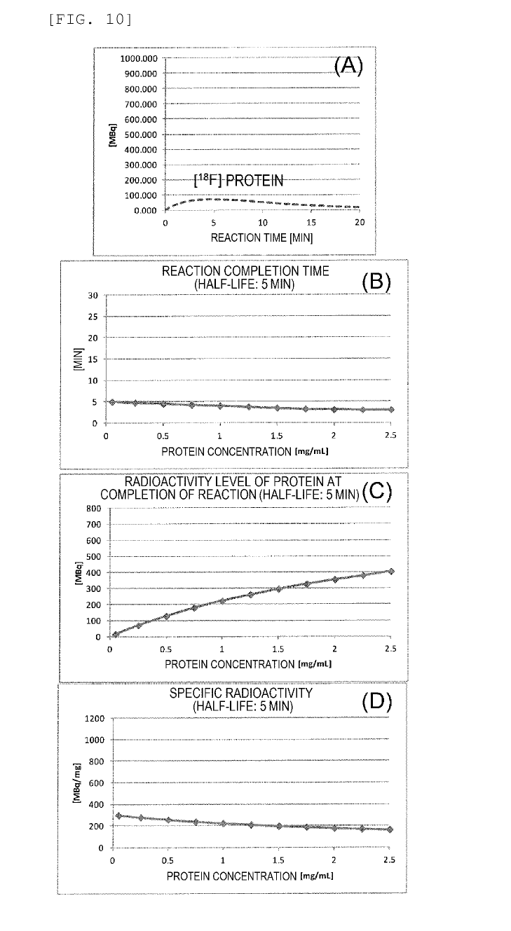

It is considered that k2 in the reaction of BSA (2.5 mg/mL) does not depend on the protein concentration and becomes constant. As a result of actually performing an experiment, k2 became about 0.12 in the reactions of BSA at 0.025, 0.25, and 2.5 mg/mL. Since the BSA concentration was increased to 10 times, k1[BSA] is as follows: k1[BSA]=0.41. By using k1[BSA]=0.41 and k2=0.125, and also using the formulae (8), (9), and (10), a relationship between a reaction time t and each of [¹⁸F]SFB [¹⁸F]BSA, and [¹⁸]FBzA was graphed as in FIG. 8. However, in the LC analysis before the reaction (reaction time: 0 min), [[¹⁸F]SFB₀] was 98.0%, and therefore, the graphs were created by changing 100 in the right side of each of the formulae (8), (9), and (10) to 98.0. Further, [[¹⁸F]FBzA₀] was 2.0% at a reaction time of 0 min, and therefore, the graph was created by adding 2.0 to the formula (10). When the experimental data obtained at reaction times of 2, 10, and 20 min in the reaction of BSA (2.5 mg/mL) were plotted on the prediction curves, it could be confirmed that the data substantially coincide with the prediction curves.

Accordingly, by calculating a reaction rate constant from the results of the LC analysis of the radioactive agent precursor before the reaction (reaction time: 0 min) and the LC analysis in the middle of the reaction, it is possible to predict a change of each component over time when the protein concentration is changed. That is, it is possible to predict a reaction completion time, a radioactivity level of a protein at completion of the reaction, and a specific radioactivity.

Next, the calculation methods for a reaction completion time, a radioactivity level of a protein component at completion of the reaction, and a specific radioactivity will be described.

When the initial radioactivity level in the formula (9) is represented by x, a relationship between a reaction time and a radioactivity level of a protein can be represented by the formula (11).

[Math. 11]

[radiolabeled proteinₜ]=x×k₁[protein]/k (1−e^{-kt})×0.5^{t/half-life} (11)

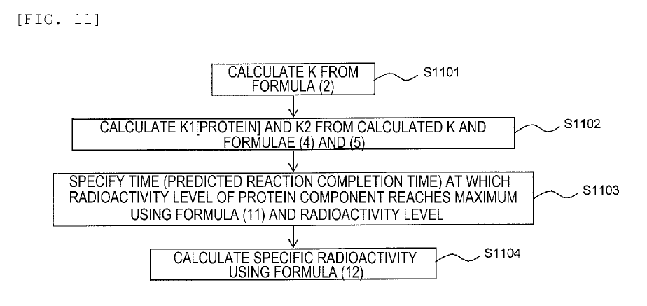

A relationship between a radiolabeled protein and a reaction time in the case where k1[protein]=0.041 and k2=0.125 when the initial radioactivity level is 1000 MBq and the protein concentration is 0.25 mg/mL is shown in FIGS. 9 (A) and 10 (A). FIG. 9 (A) shows the case where the half-life is 110 minutes, and FIG. 10(A) shows the case where the half-life is 5 minutes. When the effect of radioactive decay becomes larger than the effect of increase in radioactivity level of a protein component by a reaction, a radioactivity level of a protein component decreases as time elapses, and draws a curve with a peak at a given reaction time.

In the reaction when the half-life is 110 minutes, the highest radioactivity level of the protein component is obtained when the reaction is completed at 20 minutes, and the radioactivity level of the protein component is about 210 MBq (FIG. 9 (A)). On the other hand, in the reaction when the half-life is 5 minutes, the highest radioactivity level of the protein component is obtained when the reaction is completed at 4.75 minutes, and the radioactivity level of the protein component is about 70 MBq (FIG. 10 (A)). In this manner, it is possible to estimate the time when the reaction should be stopped and the radioactivity level of the protein component obtained at that time from the relationship between the reaction time and the radioactivity level of the protein component by using the formula (11).

Further, as described above, in the reaction in which the protein concentration is changed, by multiplying the calculated k1[protein] by a ratio of a protein concentration used for calculating k1[protein] and k2 to a desired protein concentration, k1[protein] at the desired protein concentration can be calculated. By substituting this value in the formula (11), a time at the peak top of the [radiolabeled proteinₜ] and the radioactivity level of the protein component obtained at that time are specified. For example, in the case where the value of k1[protein] is calculated from the reaction at a protein concentration of 1 mg/mL, the k1[protein] at a protein concentration of 10 mg/mL can be obtained by multiplying the k1[protein] calculated from the reaction at a protein concentration of 1 mg/mL by 10.

A relationship between a reaction completion time and a protein concentration in the case where the initial radioactivity level is 1000 MBq, the protein concentration is 0.25 mg/mL, k1[protein]=0.041, and k2=0.125 is shown in FIGS. 9(B) and 10(B). FIG. 9(B) shows the case where the half-life is 110 minutes, and FIG. 10(B) shows the case where the half-life is 5 minutes.

A relationship between a radioactivity level of a protein component at a reaction completion time and a protein concentration in the case where the initial radioactivity level is 1000 MBq, the protein concentration is 0.25 mg/mL, k1[protein]=0.041, and k2=0.125 is shown in FIGS. 9(C) and 10 (C). FIG. 9(C) shows the case where the half-life is 110 minutes, and FIG. 10(C) shows the case where the half-life is 5 minutes.

A specific radioactivity may be obtained by dividing the radioactivity level of a protein at completion of the reaction by the amount y [mg] of the protein used in the reaction. The specific radioactivity [MBq/mg] is as shown in the formula (12).

[Math. 12]

(specific radioactivity)=(radioactivity level of radiolabeled protein at completion of reaction)/y (12)

In the case where a reaction is performed at a protein concentration of 0.25 mg/mL in a synthesis volume of 1 mL, when the radioactivity level of the protein component after the reaction is predicted to be 210 MBq, the amount of the protein used in the reaction is 0.25 mg, and therefore, the specific radioactivity is calculated as follows: 210/0.25=840 [MBq/mg].

A relationship between a specific radioactivity and a protein concentration in the case where k1[protein]=0.041 and k2=0.125 when the initial radioactivity level is 1000 MBq and the protein concentration is 0.25 mg/mL is shown in FIGS. 9(D) and 10(D). FIG. 9 (D) shows the case where the half-life is 110 minutes, and FIG. 10 (D) shows the case where the half-life is 5 minutes.

### Processing of Processing Section 170

FIG. 11 shows a processing flow of a processing section 170. By using the formula (2), k is calculated from the LC analysis results at a reaction time of 0 min and at a reaction time of t min (S1101). By using the calculated k and the LC analysis results at a reaction time of t min, k[protein] and k2 are calculated from the formulae (4) and (5) (S1102). The k1[protein] when the protein concentration is obtained by multiplying a protein concentration ratio by calculated k1[protein], and k2 is constant regardless of the protein concentration. By using these values, a time (predicted reaction completion time) when the radioactivity level of the protein component when the protein concentration is changed reaches the maximum and the radioactivity level of the protein component are specified from the formula (11) (S1103). The specific radioactivity of the protein component at completion of the reaction is calculated from the formula (12) (S1104).

By the flow of these processing operations, graphs showing relationships between a protein concentration and a reaction completion time, between a protein concentration and a radioactivity level of a protein component at completion of the reaction, and between a protein concentration and a specific radioactivity can be obtained (FIGS. 9(B) to 9(D) and FIGS. 10(B) to 10(D)). By using the obtained graphs, it is possible to present a radioactive agent synthesis condition under which the highest specific radioactivity is obtained among the reaction conditions satisfying the requirement conditions for an objective substance. Further, the reaction can be stopped at an appropriate time.

For example, in the case where the actual synthesis requirements in which synthesis is performed at an initial radioactivity level of 1000 MBq and a protein component needs a radioactivity of 400 MBq or more are set, as a result of LC analysis, the graphs shown in FIGS. 9(B) to 9(D) are assumed to be obtained. From FIG. 9(C), a protein concentration at which a protein component having a radioactivity of 400 MBq or more is obtained is found to be 0.65 mg/mL or more (54 in FIG. 9(C)). Subsequently, from FIG. 9(D), among the protein concentration conditions at 0.65 mg/mL or more, a reaction condition under which a protein component having a highest specific radioactivity is obtained is a protein concentration of 0.65 mg/mL (55 in FIG. 9(D)), and the specific radioactivity at that time is 640 MBq/mg (56 in FIG. 9(D)). Further, it is predicted from FIG. 9(B) that a time when the reaction is stopped is 15 minutes (57 in FIG. 9(B)). From these predictions, it is presented to a user that when a reaction is performed at a protein concentration of 0.65 mg/mL, a protein component having a specific radioactivity of 640 MBq/mg can be synthesized at 400 MBq, and the reaction is stopped after 15 minutes from the start of the reaction, and the actual synthesis is performed.

Also in the actual synthesis, by performing LC analysis of part of the reaction mixture in the same manner as the test synthesis, the reaction rate constant is calculated, whereby a predicted reaction completion time can be calculated. According to this, it becomes possible to stop the reaction at an appropriate time with high accuracy.

### Setting of Analysis Conditions

Next, one example of a screen displayed in the display section 173 will be described.

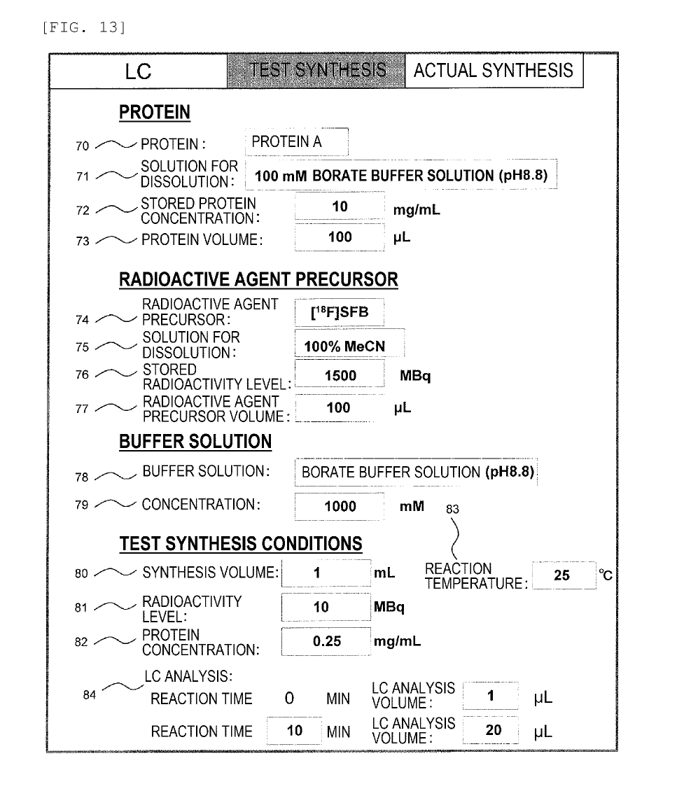

FIG. 12 shows one example of a screen for LC analysis. Here, it is possible to set the composition of the mobile phase (61), the flow rate (62), and the LC elution condition (63), and the LC analysis data are displayed on a real time basis (60). These setting data are displayed on the screen of the display section 173 by inputting from the input section 172 by a user.

Incidentally, it is possible to display a plurality of LC analysis data, and it is also possible to perform peak picking through software and to correct peak picking manually. The analysis such as peak picking may be performed on another screen. Further, as shown in FIG. 6, the area of each peak or the ratio thereof may be displayed.

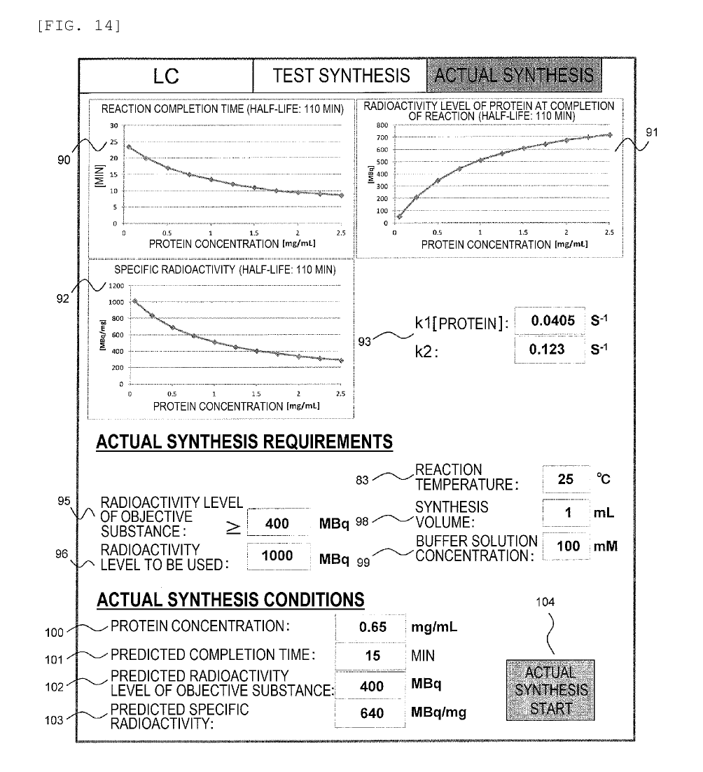

FIG. 13 shows one example of a screen for test synthesis. Here, the respective data of the name of a protein (70), the concentration and pH of a solution for dissolving the protein (71), the concentration of the protein placed in the protein solution storage section 13 (72), the volume of the protein (73), the name of a radioactive agent precursor (74), a solution for dissolving the radioactive agent precursor (75), the radioactivity level of the radioactive agent precursor placed in the radioactive agent precursor solution storage section 16 (76), the volume of the radioactive agent precursor (77), the type of a buffer solution placed in the buffer solution storage section 14 (78), and the concentration of the buffer solution (79) are input from the input section 172 and displayed on the display section 173.

The concentration of the protein is desirably higher because the concentration in the reaction is adjusted by dilution. As another method, the protein dried to a solid may be used in the reaction by dissolving it to a desired concentration. The radioactivity level is obtained by measuring it using a Curie meter or the like. Further, the radioactivity level decreases every second due to radioactive decay, and therefore is desirably displayed on a real time basis by calculation. The concentration of the buffer solution is desirably higher because the concentration in the reaction is adjusted by dilution.

As the test synthesis conditions, the synthesis volume (80), the radioactivity level to be used in test synthesis (81), the protein concentration (82), the reaction temperature (83), and for LC analysis (84), the LC analysis timing and the volume of a sample to be analyzed by LC are input. As the LC analysis timing, it is necessary to perform the LC analysis of a reaction mixture before the reaction is completed, that is, in the middle of the reaction. The LC analysis may be performed a plurality of times (for example, at reaction times of 1 min, 10 min, and 20 min). Further, by performing the LC analysis a plurality of times, a possibility that the LC analysis can be performed before the reaction is completed is increased. Further, the error of the reaction rate constant is reduced, and therefore, it is desirable to perform the LC analysis a plurality of times.

FIG. 14 shows one example of a screen for actual synthesis. Here, the values of k1[protein] and k2 (93), and the graphs showing relationships between a protein concentration and a reaction completion time (90), between a protein concentration and a radioactivity level of a protein at completion of the reaction (91), and between a protein concentration and a specific radioactivity (92) obtained as a result of the test synthesis are also displayed. As the actual synthesis conditions, the respective data of the radioactivity level to be used in actual synthesis (96), a necessary radioactivity level of a protein component (a radioactivity level of an objective substance) (95), the synthesis volume to be used in the actual synthesis (98), the concentration of a buffer solution (99), and the reaction temperature (83) are input from the input section 172 and displayed on the screen of the display section 173.

Here, in place of the synthesis volume to be used in the actual synthesis, the amount of a protein may be set. Further, in the case where there is a limit to the synthesis time, a limit may be provided for the synthesis time. Further, a specific radioactivity condition may be set. The protein concentration (100) at which the highest specific radioactivity is obtained among the protein concentration conditions satisfying the actual synthesis requirements given by a user, the predicted reaction time at that time (101), the predicted radioactivity level of the objective substance (102), and the predicted specific radioactivity (103) are displayed. It is desirable to be able to input the values for the actual synthesis requirements before the test synthesis or even after the test synthesis, and when the actual synthesis requirements are changed, it is desirable to be able to calculate the values for the actual synthesis conditions and reflect the change on a real time basis. When the user confirms that there is no problem with the actual synthesis conditions, the user pushes the actual synthesis start button (104) to start the actual synthesis. Alternatively, the device may have a function to select desired conditions from the graphs showing relationships between a protein concentration and a reaction completion time (90), between a protein concentration and a radioactivity level of a protein at completion of the reaction (91), and between a protein concentration and a specific radioactivity (92), and reflect the conditions in the actual synthesis conditions.

For example, in the case where the preparations are made as follows: the stored protein concentration (72): 10 mg/mL, the protein volume (73): 100 μL, the stored radioactivity level (76): 1500 MBq, the radioactive agent precursor volume (77): 100 μL, and the buffer solution concentration (79): 1000 mM, and the actual synthesis is performed under the following conditions : the radioactivity level to be used (96): 1000 MBq, the protein concentration (100): 0.65 mg/mL, the buffer solution concentration (99): 100 mM, and the synthesis volume (98): 1 mL, 768 μL of water is transferred to the reaction vessel 2 from the water storage section 15 by driving the syringe 10, 65 μL of the protein solution is transferred to the reaction vessel 2 from the protein solution storage section 13 by driving the syringe 8, 100 μL of the buffer solution is transferred to the reaction vessel 2 from the buffer solution storage section 14 by driving the syringe 9, 67 μL of the radioactive agent precursor solution is transferred to the reaction vessel 2 from the radioactive agent precursor solution storage section 16 by driving the syringe 11, and the reaction is performed, the radioactive agent precursor at 1000 MBq and the protein at a concentration of 0.65 mg/mL are reacted in a synthesis volume of 1 mL with the buffer solution at a concentration of 100 mM.

As described above, according to preferred embodiments, the properties of a protein can be specified. For example, a biomolecule produced for radiolabeling is subjected to the test synthesis, and the obtained reaction rate constant, and graphs showing relationships between a protein concentration and a reaction completion time, between a protein concentration and a radioactivity level of a protein at completion of the reaction, and between a protein concentration and a specific radioactivity, and optimal reaction conditions can also be sold as a data sheet along with the biomolecule.

Hereinabove, preferred embodiments have been described, however, the invention is not limited to the above-mentioned examples and can be implemented by being modified and applied in various ways. For example, the numerical values, numbers, amounts, ranges, etc. of components in the above-mentioned embodiments are examples, and other examples may be applied. Further, when referring to ranges or boundary values in the embodiments, the phrases “or more”, “or less”, etc. are not necessarily used in a mathematically strict sense as whether or not the boundary values themselves are included. The phrase “or more” or “or less” may be regarded to be the same as, for example, “exceeding a certain value or range” or “less than”.

### REFERENCE SIGNS LIST

1: temperature adjusting section, 2: reaction vessel, 3 to 7: flow channel selection valve, 8: syringe for sucking and discharging protein solution, 9: syringe for sucking and discharging buffer solution, 10: syringe for sucking and discharging water, 11: syringe for sucking and discharging radioactive agent precursor solution, 12: syringe for LC analysis, 13: protein solution storage section, 14: buffer solution storage section, 15: water storage section, 16: radioactive agent precursor solution storage section; 17: information processing device, 170: processing section, 171: memory section, 172: input section, 173: display section, 19: LC section, 20: radioactivity detection section, 54: protein concentration range capable of obtaining protein component with 400 MBq or more, 55: protein concentration at which highest specific radioactivity is obtained in protein concentration range capable of obtaining protein component with 400 MBq or more, 56: predicted specific radioactivity when performing reaction at protein concentration of 55, 57: predicted reaction time when performing reaction at protein concentration of 55, 60: LC chromatogram, 61: mobile phase composition, 62: flow rate setting, 63: LC separation conditions, 70: protein type, 71: solution for dissolving protein, 72: stored protein concentration, 73: protein volume, 74: radioactive agent precursor type, 75: solution for dissolving radioactive agent precursor, 76: stored radioactivity level, 77: radioactive agent precursor volume, 78: buffer solution type, 79: buffer solution concentration, 80: test synthesis volume, 81: radioactivity level in test synthesis, 82: protein concentration in test synthesis, 83: reaction temperature in test synthesis, 84: LC analysis conditions in test synthesis, 90: graph showing relationship between protein concentration and predicted reaction completion time, 91: graph showing relationship between protein concentration and radioactivity level of protein component at completion of reaction, 92: graph showing relationship between protein concentration and specific radioactivity of protein component at completion of reaction, 93: reaction rate constant, 95: actual synthesis requirement (radioactivity level of objective substance), 96: actual synthesis requirement (radioactivity level to be used), 98: actual synthesis requirement (synthesis volume), 99: actual synthesis requirement (buffer solution concentration), 100: actual synthesis condition (protein concentration), 101: actual synthesis condition (predicted completion time), 102: actual synthesis condition (predicted radioactivity level of objective substance), 103: actual synthesis condition (predicted specific radioactivity), 104: actual synthesis start button, 110: control section

1. A radioactive agent synthesis device, characterized by comprising:
a reaction vessel in which at least a radioactive agent precursor solution and a protein solution are placed and a synthesis reaction is performed;
a measurement section which measures the amount of a component of a radioactive agent precursor before the reaction and the amount of a component of a reaction mixture in the middle of the reaction;
a processing section which performs synthesis processing including processing in which a reaction rate constant is calculated from the measurement information of the amounts of the components, a reaction rate constant when a biomolecule concentration is changed is calculated, and a reaction time, a specific radioactivity, and a radioactivity level of an objective substance at each biomolecule concentration are calculated;
a display section which displays the result of the synthesis processing by the processing section; and
an input section from which a condition for the synthesis processing is input, wherein
the processing section uses the result of the first synthesis processing obtained by the processing section as a synthesis condition for second synthesis processing to be performed thereafter along with the condition to be input from the input section.

2. The radioactive agent synthesis device according to claim 1, wherein the processing section sets a biomolecule concentration at which the obtained radioactivity level of the biomolecule is a fixed value or more, and the calculated synthesis time as the second synthesis conditions.

3. The radioactive agent synthesis device according to claim 1, wherein the processing section sets a biomolecule concentration at which the obtained radioactivity level of the biomolecule is a fixed value or more and also the specific radioactivity reaches the maximum, and the calculated synthesis time as the second synthesis conditions.

4. The radioactive agent synthesis device according to claim 1, wherein the processing section sets a biomolecule concentration at which the specific radioactivity reaches the maximum, and the calculated synthesis time as the second synthesis conditions.

5. A radioactive agent synthesis method, which is a radioactive agent synthesis method for synthesizing a radioactive agent using a radioactive agent precursor, characterized in that
first synthesis is performed according to preset first synthesis conditions including a radioactivity level, a synthesis volume, and a protein concentration, and
the data of a specific radioactivity and a reaction completion time determined by the first synthesis are used for setting the conditions for second synthesis to be performed thereafter.

6. The radioactive agent synthesis method according to claim 5, wherein
the first synthesis includes a step of measuring the amount of a component of a radioactive agent precursor before the reaction, a step of measuring the amount of a component of a reaction mixture in the middle of the reaction, a step of calculating a reaction rate constant from the measurement information of the amounts of the components, a step of calculating a reaction rate constant when a biomolecule concentration is changed, a step of calculating a reaction time, a specific radioactivity, and a radioactivity level of an objective substance at each biomolecule concentration, and a step of selecting a reaction condition satisfying the synthesis condition, and
the second synthesis is performed under the selected reaction condition.

7. The radioactive agent synthesis method according to claim 5, wherein
the first synthesis includes:
a step of measuring the amount of a component in a radioactive agent precursor solution;
a step of performing synthesis at a predetermined radioactive agent precursor concentration and at a predetermined biomolecule concentration;
a step of measuring the amount of a component in a solution in a synthesis process;
a step of calculating a reaction rate constant from the measurement result of the amount of the component in the radioactive agent precursor solution and the measurement result of the amount of the component in the solution in the synthesis process;
a step of calculating a reaction rate constant when a biomolecule concentration is changed;
a step of calculating a function of the radioactivity level of the biomolecule and a synthesis time using the reaction rate constant when the biomolecule concentration is changed in the calculation processing section;
a step of calculating the maximum value of the radioactivity level of the biomolecule and the synthesis time therefor; and
a step of calculating a specific radioactivity from the maximum value of the radioactivity level of the biomolecule and the amount of a protein used in the synthesis.

8. The radioactive agent synthesis method according to claim 6, wherein a biomolecule concentration at which the obtained radioactivity level of the biomolecule is a fixed value or more, and the calculated synthesis time are set as the second synthesis conditions.

9. The radioactive agent synthesis method according to claim 6, wherein a biomolecule concentration at which the obtained radioactivity level of the biomolecule is a fixed value or more and also the specific radioactivity reaches the maximum, and the calculated synthesis time are set as the second synthesis conditions.

10. The radioactive agent synthesis method according to claim 6, wherein a biomolecule concentration at which the specific radioactivity reaches the maximum, and the calculated synthesis time are set as the second synthesis conditions.

11. The radioactive agent synthesis method according to claim 6, wherein in the calculation step, k is calculated using the formula (2) from the results of LC analysis at a reaction time of 0 min and a reaction time of t min, k₁[protein] and k₂ are calculated from the formulae (4) and (5) using the calculated k and the result of LC analysis at a reaction time of t min (wherein the k₁[protein] when a protein concentration is changed becomes a protein concentration ratio×calculated k₁[protein], and k₂ is constant regardless of the protein concentration), a time (a predicted reaction completion time) at which the radioactivity level of the protein component when a protein concentration is changed reaches the maximum and the radioactivity level of the protein component are calculated from the formula (11) using these values, and the specific radioactivity of the protein component at completion of the reaction is calculated from the formula (12):
[Math. 2]
k=−In[[[¹⁸F]SFBₜ]/[[¹⁸F]SFB₀]]/t (2)
[Math. 4]
[[¹⁸F]proteinₜ]/[[¹⁸F]proteinₜ]+[[¹⁸F]FBzAₜ]]=k₁[protein]/k (4)
[Math. 5]
[[¹⁸F]FBzAₜ]/[[¹⁸F]proteinₜ]+[[¹⁸F]FBzAₜ]=k₂/k (5)
[Math. 11]
[radiolabeled proteinₜ]=x×k₁[protein]/k(1−e^{-kt})×0.5^{t/half-life} (11)
[Math. 12]
(specific radioactivity)=(radioactivity level of radiolabeled protein at completion of reaction)/y (12)
